(19) United States
(12) Patent Application Publication
(10) Pub. No.: US 2023/0180795 A1
(43) Pub. Date: Jun. 15, 2023

(54) PALATABLE SUPPORT COMPOSITIONS FOR ADMINISTRATION OF MEDICINAL PRODUCTS

(71) Applicant: MARS, INCORPORATED, McLean, VA (US)

(72) Inventors: Karine BRECIN, Aimargues (FR); Sandrine VIALLE, Aimargues (FR); Melody RAASCH, Lewisburg, OH (US)

(73) Assignee: MARS, INCORPORATED, McLean, VA (US)

(21) Appl. No.: 17/997,778

(22) PCT Filed: Apr. 23, 2021
(86) PCT No.: PCT/US2021/028872

§ 371 Date: Nov. 2, 2022

(30) Foreign Application Priority Data
May 6, 2020 (EP) 20173110.6

(51) Int. Cl. A23K 20/147; A23K 50/40; A23K 10/10; A23K 20/158

(52) U.S. Cl. CPC A23K 20/147; A23K 50/40; A23K 10/10; A23K 20/158

(57) ABSTRACT

The presently disclosed subject matter relates to palatable support compositions for administering one or more medicinal products to an animal, for example, a feline. The palatable support compositions include (i) from about 20% to about 40% by weight of a humectant plasticizer; (ii) from about 0.5% to about 8% by weight of one or more gelling agents; and (iii) from about 20% to about 50% by weight of protein, on a dry matter basis. The present disclosure further provides methods for preparing and use of such palatable support compositions.

### CROSS-REFERENCE TO RELATED APPLICATIONS

This application claims the benefit of priority to EP Patent Application Serial No. 20173110.6, filed on May 6, 2020, the contents of which are incorporated herein by reference in their entirety.

### 1. FIELD

The presently disclosed subject matter relates to palatable support compositions, and in particular palatable support compositions for veterinary medication, and methods for preparing and using the same.

### 2. BACKGROUND

The ease of administering oral medication to companion animals, particularly to cats, is a major aspect of owner compliance and can have a significant impact on an animal's health. The willingness of an animal to voluntarily ingest medicine can be dependent upon a plurality of factors such as palatability, texture, size, shape and potentially color of the product. Cats are particularly reticent to get most medicine and pet owners can also be reticent to force anything onto their animal. Palatability can be considered as the primary factor in the willingness of an animal to voluntarily ingest medicine and can be determined, for example, by the smell, taste, and feeling of the medicine composition in the mouth. Usually, palatability can be achieved by adding a palatability enhancer substance or a flavoring substance to the medicine composition, such as to a chewable tablet or a soft chew composition, during its manufacturing process.

Numerous solutions have been proposed to facilitate the absorption of a medication by an animal. One technical solution includes masking the unpleasant taste or smell of a drug substance by the encapsulation or coating thereof. Another technical solution includes isolating the active substances in the center of a matrix comprising a palatable substance to mask the taste of these active substances and to facilitate the taking and consumption thereof. However, a known disadvantage of compositions in which one or more veterinary active substances are embedded within a palatable material is the potential storage instability of the active substances contained therein. With such veterinary medicine compositions, the physical and/or chemical interactions between components of the palatable matrix, particularly water and hygroscopic ingredients, and the one or more veterinary active substances can lead to an alteration of the active substances over time and thus a reduction of their therapeutic activity.

Another technical solution to facilitate the absorption of a medication by an animal includes providing a palatable support. The palatable support can be used, for example, by extemporaneously including a selected drug substance within the support prior to ingestion by the animal. Such kind of palatable support is disclosed in U.S. Patent Application Pub. No. 2009/0054536, which provides a palatable support that at least partially wraps an active substance initially independent from the support. The palatable support includes 3% to 50% w/w of glucose syrup, the dextrose equivalent (DE) thereof being in the range of 5 to 60.

European Patent No. 0 574 301 B1 discloses a palatable support in the form of a pastille for facilitating the adsorption of medicinal tablets by dogs and cats. The support includes a matrix of a palatable material including two main faces, one of which has a recess open to the outside with a form and dimensions corresponding to the tablet. When used, the tablet can be pressed into the pastille and held on this face on which it remains visible, while the other main face being essentially smooth can be presented to the animal.

U.S. Pat. No. 4,857,333 discloses an animal food product for administering medication to animals which can be a formed, chewable food treat with at least one pre-formed pocket therein. The pre-formed pocket can open to an outer surface of the treat and can be sized to retain a medicant therein.

U.S. Pat. No. 5,674,515 discloses a food item for administering a medication, particularly to a dog. The food item can be a short edible tube with an interior chamber having a closed end and open end. The medication can be inserted into the chamber through the open end of the tube and the open end of the tube can be pinched closed.

U.S. Pat. No. 5,853,757 discloses a carrier for pills, tablets, or capsules allowing for ease of oral consumption by animals. The carrier can have a pre-formed chamber allowing for the insertion of medication therein. The carrier can be deformable and can cause securement of the medication within the chamber. In some embodiments, the carrier can be approximately 66% meat by-product, approximately 17% soy flour, approximately 5% water, and approximately 7% glycerin.

U.S. Pat. No. 6,143,316 discloses a digestible pouch for aiding in the oral administration of substances suitable for ingestion of medicinal agents. The pouch can be formed of an edible food stuff material pleasing to the recipient, such as a domestic animal. The pouch can include an inner compartment area for housing the particular substance prior to oral administration.

PCT Application Pub. No. WO 2003/030863 discloses a dough wrap for a pill, tablet, or capsule. The dough includes at least one flour, at least one soluble fiber, at least one oil, and at least one other pharmaceutically acceptable agent. The dough can be capable of being molded around the selected medicament.

PCT Application Pub. No. WO 1995/020942 discloses a lure for facilitating the administration of drugs to animals, particularly to dogs, including a body made of palatable material and shaped like a conventional sugar lump. The body includes a cavity open on one side thereof and cam be shaped to receive a medicinal product in solid dosage form. In some embodiments, the lure includes a combination of pork liver powder, cocoa flavor, magnesium stearate, and mainly sorbitol.

The numerous technical solutions illustrate the complexity linked to the conception of palatable compositions comprising one or more veterinary active substances. This complexity is particularly increased for veterinary compositions suitable for preventing or treating physiological disorders or diseases in felines, particularly in cats. Due to the finicky nature of the domestic cats' feeding habits, palatability is a highly complex area of feline nutrition. However, relatively little is known about the specific compounds and ingredients responsible for the palatability of cat foods. Cats are considered to prefer certain amino acids (See, e.g., Zaghini and Biagi, 2005, Veterinary Research Communications, Vol. 29: 39-44). However, a recent study by Savolainen, et al. (2019, Veterinary and Animal Science, Vol. 7: 100054), which used a realistic method of palatability testing, showed significant differences were detected between a tablet comprising palatable synthetic flavors (i.e., a serial of 18 tested amino acids) and the same tablet devoid of synthetic flavor used as a placebo. As illustrated above, there is currently deficient knowledge relating to palatable veterinary compositions for cats. Thus, as evidenced in the recent literature, conceiving of palatable materials for administering medicinal products to felines, particularly to cats, remains largely empirical. The skilled artisan would appreciate that references existing on the subject matter cannot simply be combined so as to form a basis towards conceiving further palatable material for veterinary medical use.

Accordingly, there remains a need for palatable support compositions that can be used for felines, particularly for cats, with extemporaneously embedding one or more veterinary active substances therein. The presently disclosed subject matter addresses these and other needs.

### 3. SUMMARY OF THE INVENTION

The presently disclosed subject matter provides for palatable support compositions, and in particular palatable support compositions for veterinary medication, and methods for preparing and using the same. In certain aspects, the palatable support compositions can be used for administering one or more medicinal products to an animal, for example, to felines such as cats. Palatable support compositions in accordance with the present disclose can advantageously have a texture resulting from good moldability properties and balanced chewiness properties. In certain aspects, the disclosed palatable support compositions can be easily handled by an owner and can readily incorporate one or more medicinal products therein. Such palatable support compositions including medicinal products can be readily accepted by animals, for example, felines such as cats.

The present disclosure relates to a palatable support for administering one or more medicinal products to a feline, including (i) from about 20% to about 40% by weight of a humectant plasticizer; (ii) from about 0.5% to about 8% by weight of one or more gelling agents; and (iii) from about 20% to about 50% by weight of protein, the weight percentage being expressed on a dry matter basis, the palatable support having a moisture content ranging from about 20% to about 35% as fed.

In some embodiments, the palatable support can include from about 1% to about 8% by weight of one or more gelling agents.

In some embodiments, the palatable support can include from about 25% to about 50% by weight of protein.

In some embodiments, the humectant plasticizer can be glycerin.

In some embodiments, the palatable support can have a hardness value from about 0.2 kg to about 1.0 kg and a cohesiveness value from about 0.1 to about 0.5.

In some embodiments, the palatable support can have a trapezoidal cylindrical shape with a bottom end and an upper end, which can have a height from about 5 mm to about 10 mm, a diameter from about 6 mm to about 12 mm at the bottom end and a diameter from about 5 mm to about 10 mm at the upper end.

In some embodiments, the palatable support can have a weight from about 0.5 grams to about 2.0 grams.

In some embodiments, the one or more gelling agents can be selected from gum Arabic, xanthan gum, guar gum, locust bean gum, carob gum, carrageenan, or a combination thereof.

In some embodiments, the palatable support can further include fat. Fat can be present in an amount from about 7.5% to about 20% by weight, on a dry matter basis. In some embodiments, fat can be present in an amount from about 10% to about 20% by weight, on a dry matter basis.

In some embodiments, protein can originate from one or more protein sources selected from chicken, poultry, fish, or a combination thereof.

In some embodiments, the palatable support can further include one or more of the following substances:

i) from about 1% to about 8% by weight, on a dry matter basis, of yeast extract;

ii) from about 1% to about 8% by weight, on a dry matter basis, of one or more palatability enhancers; and

iii) from about 1% to about 8% by weight, on a dry matter basis, of corn syrup solids.

In some embodiments, the palatable support can be a functional supplement.

In some embodiments, the feline can be a cat.

In another aspect, the present disclosure pertains to a kit of parts including (i) a palatable support as described herein and (ii) one or more medicinal product dosage units.

The present disclose further relates to a kit of parts including (i) a palatable support as described herein and (ii) one or more medicinal products.

The present disclosure further relates to the use of a palatable support as described herein for facilitating a feline medication, particularly a cat medication.

The present disclosure further relates to the use of a palatable support as described herein for administering one or more medicinal products to a feline.

The present disclosure further relates to a method of use of a palatable support as described herein for administering a medicinal product to a feline, in which the method includes:

a) selecting one or more medicinal products to be administered to the feline;

b) incorporating the one or more medicinal products in the palatable support, whereby an incorporated medicinal product is provided; and

c) providing the incorporated medicinal product obtained at step b) to the feline.

The present disclosure further relates to a method of use of a palatable support as described herein for administering a medicinal product to a feline, in which the method includes:

a) selecting one or more medicinal products to be administered to the feline;

b) incorporating the one or more medicinal products in the palatable support, so as to provide an incorporated medicinal product; and

c) providing the incorporated medicinal product obtained at step b) to the feline.

The palatable product can include (i) from about 20% to about 40% by weight of a humectant plasticizer; (ii) from about 0.5% to about 8% by weight of one or more gelling agents; and (iii) from about 20% to about 50% by weight of protein, the weight percentage being expressed on a dry matter basis, the palatable support having a moisture content ranging from about 20% to about 35% as fed.

In some embodiments, the palatable support for use according to the present disclosure can include from about 1% to about 8% by weight of one or more gelling agents.

In some embodiments, the palatable support for use according to the present disclosure can include from about 25% to about 50% by weight of protein.

The present disclosure further relates to a method including providing a palatable support for administering one or more medicinal products to a feline, the palatable support including (i) from about 20% to about 40% by weight humectant plasticizer; (ii) from about 0.5% to about 8% by weight of one or more gelling agents; and (iii) from about 20% to about 50% by weight of protein, the weight percentage being expressed on a dry matter basis, the palatable support having a moisture content ranging from about 20% to about 35% as fed.

The present disclosure further relates to a method for administering one or more medicinal products to a feline, the method including at least a step of providing a palatable support comprising (i) from about 20% to about 40% by weight humectant plasticizer; (ii) from about 0.5% to about 8% by weight of one or more gelling agents; and (iii) from about 20% to about 50% by weight of protein, the weight percentage being expressed on a dry matter basis, the palatable support having a moisture content ranging from about 20% to about 35% as fed.

The present disclosure further provides a palatable support composition for administering one or more medicinal products to a feline. The palatable support composition includes from about 20% to about 40% by weight of a humectant plasticizer; from about 0.5% to about 8% by weight of one or more gelling agents; and from about 20% to about 50% by weight of protein, the weight percentage on a dry matter basis. The palatable support composition has a moisture content from about 20% to about 35% as fed.

In certain embodiments, the humectant plasticizer can be glycerin.

In certain embodiments, the composition can have a hardness value from about 0.2 kg to about 1.0 kg and a cohesiveness value from about 0.1 to about 0.5 in a compression TPA test.

In certain embodiments, the composition can have a trapezoidal cylindrical shape. The shape can have a bottom end and an upper end; a height from about 5 mm to about 10 mm; a diameter from about 6 mm to about 12 mm at the bottom end; and a diameter from about 5 mm to about 10 mm at the upper end.

In certain embodiments, the composition can have a weight from about 0.5 grams to about 2.0 grams.

In certain embodiments the one or more gelling agents can include gum Arabic, xanthan gum, guar gum, locust bean gum, carob gum, carrageenan, or a combination thereof.

In certain embodiments, the composition can further include fat in an amount from about 7.5% to about 20% by weight, on a dry matter basis.

In certain embodiments, the protein can originate from one or more protein sources including chicken, poultry, fish, or a combination thereof.

In certain embodiments, the composition can include one or more of from about 1% to about 8% by weight yeast extract, on a dry matter basis; from about 1% to about 8% by weight one or more palatability enhancers, on a dry matter basis; and from about 1% to about 8% by weight corn syrup solids, on a dry matter basis.

In certain embodiments, the composition can be a functional supplement.

The present disclosure further provides a kit. The kit includes a palatable support composition in accordance with the present disclosure and one or more medicinal product dosage units.

The present disclosure further relates to the use of a palatable support composition for facilitating a feline medication and for administering one or more medicinal products to a feline.

The present disclosure further provides a method of use of a palatable support composition for administering one or more medicinal products to a feline. The method includes selecting one or more medicinal products to be administered; incorporating the one or more medicinal products in the palatable support composition to provide an incorporated medicinal product; and providing the incorporated medicinal product to the feline.

The present disclosure further provides a method of use of a palatable support composition for administering one or more medicinal products to a feline. The method includes selecting one or more medicinal products to be administered; incorporating the one or more medicinal products in the palatable support to provide an incorporated medicinal product; and providing the incorporated medicinal product to the feline. The palatable support composition includes from about 20% to about 40% by weight humectant plasticizer; from about 0.5% to about 8% by weight of one or more gelling agents; and from about 20% to about 50% by weight of protein, the weight percentage being expressed on a dry matter basis. The palatable support composition has a moisture content ranging from about 20% to about 35% as fed.

The present disclosure further provides a method including providing a palatable support composition for administering one or more medicinal products to a feline. The palatable support composition includes from about 20% to about 40% by weight humectant plasticizer; from about 0.5% to about 8% by weight of one or more gelling agents; and from about 20% to about 50% by weight of protein, the weight percentage being expressed on a dry matter basis. The palatable support has a moisture content ranging from about 20% to about 35% as fed.

The present disclosure further provides a method for administering one or more medicinal products to a feline. The method includes providing a palatable support composition including from about 20% to about 40% by weight humectant plasticizer; from about 0.5% to about 8% by weight of one or more gelling agents; and from about 20% to about 50% by weight of protein, the weight percentage being expressed on a dry matter basis. The palatable support has a moisture content ranging from about 20% to about 35% as fed.

In certain embodiments, the feline can be a cat.

The present disclosure further provides a method for preparing a palatable support composition. The method includes providing a blend of a plurality of dry ingredients including proteins; providing an aqueous solution including a humectant plasticizer; providing a fat mixture, comprising a plurality of fat-based ingredients from animal and/or vegetable origin; mixing the blend of the plurality of dry ingredients, the aqueous solution, and the fat mixture, and heating the resulting mixture to a temperature of at least about 80° C. to provide a cooked dough; cooling the cooked dough; and molding the cooled dough to provide the palatable support composition.

### 4. DETAILED DESCRIPTION

The presently disclosed subject matter relates to improved palatable support compositions and methods of making and using the same. In certain embodiments, such palatable support compositions can include a humectant plasticizer, one or more gelling agents, and protein. Palatable support compositions in accordance with the present disclosure can be used for veterinary medication, for example, in the administration of one or more medicinal products to an animal, for example, to felines such as cats.

For clarity and not by way of limitation, this detailed description is divided into the following sub-portions:

4.1. Definitions;

4.2. Palatable support compositions;

4.3. Methods of making palatable support compositions;

4.4. Methods of using palatable support compositions;

4.5. Features of palatable support compositions; and

4.6. Applications and kits.

### 4.1. Definitions

The terms used in this specification generally have their ordinary meanings in the art, within the context of this disclosure and in the specific context where each term is used. Certain terms are discussed below, or elsewhere in the specification, to provide additional guidance in describing the compositions and methods of the disclosure and how to make and use them.

As used herein, “about” or “approximately” means within an acceptable error range for the particular value as determined by one of ordinary skill in the art, which will depend in part on how the value is measured or determined, i.e., the limitations of the measurement system. For example, “about” can mean a range of up to 20%, preferably up to 10%, more preferably up to 5%, and more preferably still up to 1% of a given value.

The term “animal protein” as used herein refers to proteins that originate from vertebrates, such as mammals, poultry and fish. Animal protein can originate, for example, from muscle meat, organs, tendons, or bone.

The term “antioxidant” as used herein refers a substance or a component that is capable of reacting with free radicals and neutralizing them.

The expression “as fed” as used herein refers to a method of expressing the concentration of a nutrient or a component in a feed by expressing its concentration in the state it is fed, which includes moisture.

The term “Aw” as used herein refers to water activity.

The expression “dry matter (DM) basis” as used herein refers to a method of expressing the concentration of a nutrient or a component in a feed by expressing its concentration relative to its dry matter content, i.e., the concentration remaining once the moisture has been taken out.

The term “fat” as used herein refers to lipidic compounds, fatty acids and esters of fatty acids such as triglycerides, diglycerides, monoglycerides as well as phospholipids. The expressions “fat” or “source of fat” or “fat source” as used herein refer to any food-acceptable fat(s) and/or oil(s) irrespective of their consistency at room temperature, i.e., irrespective whether said “source of fat” is present in essentially fluid form or in essentially solid from.

As used herein, the term “glycerin” as used herein refers to its conventional meaning in the art. Glycerin, which can also be termed “glycerol” herein, is the simple polyol 1,2,3-propanetriol.

The term “humectant plasticizer” as used herein refers to any humectant plasticizers that have humectant properties and that are compatible with food compositions. Plasticizer properties allow to bring textural properties to the composition in which it is added. Humectant properties allow the binding with water and therefore reducing the water activity (Aw) of composition.

The term “non-animal protein” as used herein refers to proteins that are not animal protein. Examples of non-animal protein include vegetable proteins, algal proteins, egg proteins, milk proteins, microbial proteins, and insect proteins.

The term “palatability” as used herein refers to a relative preference of an animal for one ingestible product to another. Palatability refers to the overall willingness of an animal to eat a certain ingestible product. Advantageously but not necessarily, palatability further refers to the capacity of the eaten ingestible product to satisfy the animal. Whenever an animal shows a preference, for example, for one of two or more ingestible products, the preferred product is more “palatable”, and has “enhanced palatability”, The relative palatability of one ingestible product compared to one or more other ingestible products can be determined, for example, in side-by-side, free-choice comparisons, e.g., by relative consumption of the ingestible products, or other appropriate measures of preference indicative of palatability. It can advantageously be determined by a standard testing protocol in which the animal has equal access to both ingestible products such as a test called “two-bowl test” or “versus test” (see below). Such preference can wise from any of the animal's senses, but typically is related to, inter alia, taste, aftertaste, smell, mouth feel and/or texture.

The term “palatability enhancer” as used herein refers to any compound, composition, formulation, or other material useful for enhancing the attractiveness and palatability of a comestible composition such as a food composition, supplement, medicament, or the like. Thus, such palatability enhancer can contribute to initial appeal, continued consumption, or repeated presentation aspects of palatability, or any combination thereof. Such product can consist of liquid and/or powder palatants. Examples fats and oils (e.g., poultry fat, tallow), flavors, chemical molecules (e.g., 2,5 dimethylpyrazine), aromas, extracts (e.g., yeast), digests, hydrolysates (e.g., poultry liver), protein ingredient (e.g., poultry meal), carbohydrate food (e.g., rice flour), powders and the like. For example, the palatability enhancer can consist of fragrances such as food fragrances, odour masking agents, and mixtures thereof, such as flavor compound as well as precursors for the above. The palatability enhancer does not consist of a food product or of a nutritional product.

The term “pet” as used herein refers to a domestic animal, including companion animals such as domestic cats and dogs.

The term “starch” as used herein refers to a polysaccharide that is composed of amylose and amylopectin.

### 4.2. Palatable Support Compositions

In certain embodiments, a palatable support composition is provided. The palatable support composition can include one or more humectant plasticizers, one or more gelling agents, one or more protein sources, one or more fat sources, and one or more further ingredients including yeast extracts, palatability enhancers, corn syrup solids, aroma agents, health aids, or medicinal products. Palatability support compositions in accordance with the present disclosure can be used for veterinary purposes in animals, for example, in felines. In particular embodiments, the palatable support can be used for administering one or more medicinal products to animals in need thereof such as felines. In certain aspects, the present disclosure relates to a palatable support for administering one or more medicinal products to a feline animal in need thereof.

As shown in the Examples, a palatable support according to the present disclosure can have a texture resulting from an optimal equilibrium between (i) good moldability properties; and (ii) balanced chewiness properties. As such, a palatable support as disclosed herein (i) can be easily handled by a feline owner, in particular by a cat owner; (ii) can allow for easy incorporation of one or more medicinal products therein; and (iii) can be easily accepted by the feline, particularly by a cat.

### 4.2.1. Humectant Plasticizers

In certain embodiments, the palatable support composition can include one or more humectant plasticizers. The humectant plasticizer can include glycerin (glycerol) or any derivative thereof. In various embodiments, the one or more humectant plasticizers can be polyols. In certain embodiments, the humectant plasticizers can be selected from glycerin (glycerol), sorbitol, propylene glycol, butylene glycol, poly-dextrose, molasses, or combinations thereof. In particular embodiments, the humectant plasticizer can be glycerin (glycerol). In certain embodiments, the humectant plasticizer can include a combination of glycerin with any other non-toxic compound acting as a plasticizer, for example, another non-toxic compound allowing to bring textural properties to a composition to which it is added and/or another non-toxic compound acting as a humectant (i.e., another non-toxic compound binding with water and therefore reducing the water activity (Aw) of a composition and a food product).

In certain embodiments, the palatable support composition can include from about 20% to about 40%, from about 20% to about 30%, from about 25% to about 35%, from about 30% to about 40%, or from about 20% to about 30% by weight of one or more humectant plasticizers, on a dry matter basis. In certain embodiments, the palatable support composition can include about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39% or about 40% by weight of one or more humectant plasticizers, on a dry matter basis.

### 4.2.2. Gelling Agents

In certain embodiments, the palatable support composition can include one or more gelling agents. The one or more gelling agents can be selected from gum Arabic, xanthan gum, guar gum, locust bean gum, carob gum, carrageenan, or combinations thereof.

In certain embodiments, the palatable support composition can include from about 0.5% to about 8%, from about 0.5% to about 6%, from about 1% to about 5%, from about 1% to about 3%, or from about 4% to about 8% by weight of one or more gelling agents, on a dry matter basis. In certain embodiments, the palatable support composition can include about 0.5%, about 1%, about 1.5%, about 2%, about 2.5%, about 3%, about 3.5%, about 4%, about 4.5%, about 5%, about 5.5%, about 6%, about 6.5%, about 7%, about 7.5% or about 8% by weight of one or more gelling agents, on a dry matter basis.

### 4.2.3. Protein Sources

In certain embodiments, the palatable support composition can include one or more protein sources or a plurality of proteins that can be contained in one or more protein sources. For example, the one or more protein sources can be in a native form, i.e., in a non-hydrolyzed form. In certain embodiments, the one or more protein sources can be present in an at least partially hydrolyzed form, which encompasses a protein which is almost completely hydrolyzed. In certain embodiments, the palatable support composition can incorporate one or more protein sources in the form of meat or animal derived material such as beef, chicken, turkey, lamb, fish, blood plasma, marrow bone, etc. or one or more thereof. In certain embodiments, the palatable support composition can be meat-free. In particular embodiments, the palatable support composition can include a meat substitute protein source such as soya, maize, gluten, or any other protein-containing soya product to provide a protein source. In certain embodiments, the palatable support composition can include additional protein sources such as soya protein concentrate, milk proteins, gluten, etc. In certain embodiments, the palatable support composition can include one or more protein sources including chicken, poultry, fish, or a combination thereof. A person skilled in the art will appreciate a wide variety of protein sources are suitable for use with the present disclosure.

In certain embodiments, the palatable support composition can include from about 20% to about 50%, from about 25% to about 50%, from about 30% to about 40%, or from about 25% to about 30% by weight of one or more protein sources, on a dry matter basis. In certain embodiments, the palatable support composition can include about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49% or about 50% by weight of one or more proteins sources, on a dry matter basis.

### 4.2.4. Fat Sources

In certain embodiments, the palatable support composition can include one or more fat sources. In certain embodiments, the one or more fat sources can include fat of animal and/or vegetable origin. Fat can be supplied by any of a variety of sources known by those skilled in the art. Plant fat sources can include, by way of example and without limitation, wheat, sunflower, safflower, rapeseed, olive, borage, flaxseed, coprah, peanuts, blackcurrant seed, palm tree, cottonseed, wheat, germ, corn germ as well as oils derived from these and other plant fat sources. Animal fat sources can include, by way of example and without limitation, chicken fat, turkey fat, beef fat, duck fat, pork fat, lamb fat, etc., fish oil or any meat, meat by-products, seafood, dairy, eggs, etc. Fat content of foods can be determined by any number of methods known by those of skill in the art. A person skilled in the art will appreciate a wide variety of fat sources are suitable for use with the present disclosure.

In certain embodiments, the palatable support composition can include from about 7.5% to about 20%, from about 10% to about 20%, from about 15% to about 20%, or from about 10% to about 15% by weight, of one or more fat sources, on a dry matter basis. In certain embodiments, the palatable support composition can include about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5% or about 20% of one or more fat sources, on a dry matter basis.

### 4.2.5. Further Ingredients

In certain embodiments, the palatable support composition can include one or more further ingredients. The one or more further ingredients can include yeast extract, palatability enhancers, corn syrup (e.g., corn syrup solids), aroma agents, health aids, medicinal products, or combinations thereof. The health aids can include, for example, vitamin(s), minerals, antioxidants(s), probiotics, prebiotics, specific health nutrients (e.g., taurine, green lipped mussel, chondroitin, collagen, polyphenols, flavonoids, curcuminoids, aloe vera extract, etc.), or amino acids. The medicinal products can include a pill, a tablet, a capsule, a liquid formulation, a paste and a gel, or a combination thereof. In certain embodiments, the medicinal products can include medicinal products having a repellent taste for animals such as cats or a general unattractive flavor, for example, enrofloxacin (bitter taste), cyclosporin. The medicinal products can include medicines used for long-terms affections such as Locox (arthritis), Zentonyl (hepatic disease), Milbemax (dewormer), or other pathologies such as Prifinial® and Emeprid®.

### 4.2.5.1. Yeast Extract

In certain embodiments, the palatable support composition includes yeast extract. Yeast extract is a well-known ingredient of human food and non-human food and is commercially available. A person skilled in the art will appreciate a wide variety of forms of yeast extract are suitable for use with the present disclosure.

In certain embodiments, the palatable support composition can include from about 1% to about 8%, from about 1% to about 5%, from about 4% to about 8%, or from about 1% to about 2% by weight of yeast extract, on a dry matter basis. In certain embodiments, the palatable support composition can include about 1%, about 1.5%, about 2%, about 2.5%, about 3%, about 3.5%, about 4%, about 4.5%, about 5%, about 5.5%, about 6%, about 6.5%, about 7%, about 7.5% or about 8% by weight of yeast extract, on a dry matter basis.

### 4.2.5.2. Palatability Enhancers

In certain embodiments, the palatable support composition can include one or more palatability enhancers. Palatability enhancers are commercially ready-to-use preparations that aim at boosting the palatability of a food composition in which it is included. A person skilled in the art will appreciate a wide variety of palatability enhancers are suitable for use with the present disclosure.

In certain embodiments, the palatable support composition can include from about 1% to about 8%, from about 1% to about 4%, from about 2% to about 5%, or from about 5% to about 8% by weight of one or more palatability enhancers, on a dry matter basis. In certain embodiments, the palatable support composition can include about 1%, about 1.5%, about 2%, about 2.5%, about 3%, about 3.5%, about 4%, about 4.5%, about 5%, about 5.5%, about 6%, about 6.5%, about 7%, about 7.5% or about 8% by weight of one or more palatability enhancers, on a dry matter basis.

### 4.2.5.3. Corn Syrup

In certain embodiments, the palatable support composition can include corn syrup. Corn syrup is a well-known ingredient used for manufacturing human food and non-human food compositions and is commercially available. Corn syrup can be in either liquid or solid form. In certain embodiments, the corn syrup can be present in a solid form, i.e., as corn syrup solids.

In certain embodiments, the palatable support composition can include from about 1% to about 8%, from about 1% to about 3%, from about 1% to about 2.5%, from about 4% to about 8%, or from about 1% to about 5% of corn syrup solids, on a dry matter basis. In certain embodiments, the palatable support composition can include about 1%, about 1.5%, about 2%, about 2.5%, about 3%, about 3.5%, about 4%, about 4.5%, about 5%, about 5.5%, about 6%, about 6.5%, about 7%, about 7.5% or about 8% by weight of corn syrup solids, on a dry matter basis.

### 4.2.5.4. Aroma Agents

In certain embodiments, the palatable support composition can include one or more aroma agents. A large number of aroma agents for food, including for cat food, are known from the skilled artisan, that can also be used in a palatable support according to the present disclosure. Thus, a person skilled in the art will appreciate a wide variety of aroma agents are suitable for use with the present disclosure. The one or more aroma agents can be selected from meat, salmon, beef, chicken, turkey, fish, cheese, other animal-based flavor agents, or a combination thereof. In certain embodiments, the aroma agent can include the protein source itself, for example, for a cat which is an obligate carnivore.

### 4.2.5.5. Health Aids

In certain aspects, the palatable support composition as disclosed herein can be used as a food supplement beyond its use for facilitating the oral administration of one or more medicinal products to a feline in need thereof. Thus, in certain embodiments, the palatable support composition can include one or more ingredients that can be useful for maintaining the recipient's health, such as vitamin(s), minerals, antioxidants(s), probiotics, prebiotics, specific health nutrients (e.g., taurine, green lipped mussel, chondroitin, collagen, polyphenols, flavonoids, curcuminoids, aloe vera extract, etc.), or amino acids. In such embodiments, since the palatable support also has recipient's health maintenance properties in addition to its ability to facilitate administration of one or more medicinal products, the palatable support composition can also be termed a “functional” supplement as used herein.

In certain embodiments, the palatable support composition can include one or more vitamins. Vitamins can be selected from vitamin A, vitamin B 1, vitamin B2, vitamin B6, vitamin B12, vitamin C, vitamin D, vitamin E, vitamin K, biotin, folic acid, inositol, niacin and pantothenic acid, or a combination thereof. A person skilled in the art will appreciate a wide variety of vitamins are suitable for use with the present disclosure.

In certain embodiments, the palatable support composition can include one or more antioxidants. Illustrative examples of such substances include, for example and without limitation, carotenoids, including beta-carotene, lycopene and lutein, selenium, coenzyme Q10 (ubiquinone), tocotrienols, soy isoflavones, S-adenosylmethionine, glutathione, taurine, N-acetylcysteine, lipoic acid and L-carnitine. A person skilled in the art will appreciate a wide variety of antioxidants are suitable for use with the present disclosure.

In certain embodiments, the palatable support composition can include one or more minerals. Minerals can be selected in a group comprising calcium, phosphorus (e.g., under the form of phosphoric acid), sodium, chloride, potassium, magnesium, oligo elements, and combinations thereof. A person skilled in the art will appreciate a wide variety of minerals are suitable for use with the present disclosure.

### 4.2.5.6. Medicinal Products

In certain embodiments, any medicinal product that can be suitable for an animal such as a feline can be incorporated into palatable support compositions of the present disclosure. In certain embodiments, the one or more medicinal products can be selected from a pill, a tablet, a capsule, a liquid formulation, a paste and a gel, or a combination thereof. In certain embodiments, the one or more medicinal products can have a repellent taste, for example, for cats or a general unattractive flavor including, for example, enrofloxacin (bitter taste), cyclosporin. In certain embodiments, the one or more medicinal product can include medicines that are used for long-terms affections, for example, Locox (arthritis), Zentonyl (hepatic disease), Milbemax (dewormer), or for other pathologies like Prifinial® and Emeprid®.

### 4.2.6. Palatable Support Compositions

In certain embodiments, the palatable support composition can include from about 20% to about 40% by weight of a humectant plasticizer, from about 0.5% to about 8% by weight of one or more gelling agents, and from about 20% to about 50% by weight of protein, the weight percentage being expressed on a dry matter basis.

In certain embodiments, the palatable support composition can include from about 20% to about 40% by weight of a humectant plasticizer, from about 1% to about 8% by weight of one or more gelling agents, and from about 25% to about 50% by weight of protein, the weight percentage being expressed on a dry matter basis. In certain embodiments, the palatable support composition can include from about 20% to about 30% by weight of a humectant plasticizer, from about 1% to about 3% by weight of one or more gelling agents, and from about 25% to about 30% by weight of protein, the weight percentage being expressed on a dry matter basis. The humectant plasticizer can be glycerin. The protein can include animal and vegetable proteins. In certain embodiments, the palatable support composition can further include corn syrup solids in an amount of from about 1% to about 3% by weight, on a dry matter basis. In certain embodiments, the palatable support composition can further include one or more palatability enhancers in an amount of about 1% to about 4% by weight, on a dry matter basis. In certain embodiments, the palatable support composition can further include yeast extract in an amount of from about 1% to about 5% by weight, on a dry matter basis. In certain embodiments, the palatable support composition can further include fats (e.g., vegetable fats), preservatives, antioxidants, prebiotics, vitamins, an acidifier, cereals (flour), water and steam condensates, and combinations thereof.

### 4.3. Methods of Making Palatable Support Compositions

In certain embodiments, a method for preparing a palatable support composition is provided. The method can include a) providing a blend of a plurality of dry ingredients and including proteins; b) providing an aqueous solution including a humectant plasticizer; c) providing a fat mixture, composed of a plurality of fat-based ingredients, from animal and/or vegetable origin; d) mixing the blend of ingredients provided at step a) with the aqueous solution provided at step b) and with the fat mixture provided at step c), and cook the resulting mixture at a temperature of about 80° C. or more, whereby a cooked dough is provided; e) cooling the cooked dough obtained at step d); and f) molding the cooled dough obtained at step e), so as to obtain a palatable support in the desirable format.

In certain embodiments, the method for preparing a palatable support composition can include a) providing a blend of a plurality of dry ingredients and including proteins, with optionally one or more additional ingredients selected from corn syrup solids, preservatives, antioxidants, gelling agents, acidifiers, prebiotics, vitamins, yeast extract and palatability enhancers, or a combination thereof; b) providing an aqueous solution including a humectant plasticizer, an aqueous solution including glycerin and water; c) providing a fat mixture, composed of a plurality of fat-based ingredients, from animal and/or vegetable origin; d) mixing the blend of ingredients provided at step a) with the aqueous solution provided at step b) and with the fat mixture provided at step c), and cook the resulting mixture at a temperature of about 80° C. or more or at a temperature of 90° C. or more, whereby a cooked dough is provided; e) cooling the cooked dough obtained at step d); and f) molding the cooled dough obtained at step e), so as to obtain a palatable support in the desirable format.

In certain embodiments, the cooking step d) can be performed at a temperature lower than about 120° C. In certain embodiments, the cooled dough obtained at step e) can be molded at step f) by pumping the said cooled dough into individual molds having approximately the size, dimensions and predetermined shape of the palatable support.

### 4.4. Methods of Using Palatable Support Compositions

In certain aspects, the present disclosure relates to the use of a palatable support composition as described herein for facilitating a feline medication, for example, by incorporating one or more medicinal products into the palatable support composition.

In certain embodiments, the present disclosure relates to administering a medicinal product to a feline. In certain embodiments, the owner can first incorporate, e.g., wrap, the medicinal product (e.g., a tablet, a capsule, a paste or a gel) in the palatable support by deforming the support so as to incorporate the medicinal product in the mass formed by the deformed palatable support. The moldable palatable support can be handled with one hand, e.g., the hand that touched the medicine in certain embodiments is not used to wrap and close the mass around it. Then, the incorporated, e.g., wrapped, medicinal product can be provided directly to the feline (e.g., cat), either by hand, on the floor, in a bowl, or on any suitable surface. Alternatively, once the medicinal product has been incorporated, e.g., wrapped, by deformation of a palatable support disclosed herein, the resulting incorporated material, e.g., wrapped material, can be mixed with the animal food, so as to be picked up by the feline when feeding. In embodiments in which the medicinal is a gel, the palatable support can be reclosed around a drop of gel, for example, if the amount of gel is low enough to fit inside the pre-made hole, when present at the upper end of the support.

In certain aspects, the present disclosure relates to a method of use of a palatable support composition as described herein as a means for administering a medicinal product to a feline. The method can include a) selecting one or more medicinal products to be administered to the feline; b) incorporating the one or more medicinal products in a palatable support as described herein, whereby an incorporated medicinal product is provided; and c) providing the incorporated medicinal product obtained at step b) to the feline.

In another aspect, the present disclosure relates to a method of use of a palatable support composition as described herein for administering one or more medicinal products to a feline. The method can include a) selecting one or more medicinal products to be administered to the feline; b) incorporating the one or more medicinal products in a palatable support as described herein, whereby an incorporated medicinal product is provided; and c) providing the incorporated medicinal product obtained at step b) to the feline.

In another aspect, the present disclosure relates to a method for administering a medicinal product to a feline. The method can include a) selecting one or more medicinal products to be administered to the feline; b) wrapping at least partially the medicinal product with a palatable support, so as to provide a wrapped medicinal product; and c) providing the wrapped medicinal product to said feline, wherein the palatable support is as disclosed herein.

In certain embodiments, at step b), incorporating the one or more medicinal products in a palatable support can be performed, for example, by wrapping at least partly the said one or more medicinal products in the palatable support.

### 4.5. Features of Palatable Support Compositions

In certain aspects, the palatable support compositions of the present disclosure can have certain features, for example, texture, size, dimensions, weight, palatability, and moisture content.

### Texture (Hardness and Cohesiveness)

The palatable support composition according to the present disclosure can have a relatively soft texture and can be easily molded around one or more medicinal products, for example, to be administered to a feline in need thereof.

In certain aspects, the palatable support composition can be defined through its textural properties, such as hardness and cohesiveness. According to the present disclosure, the hardness and cohesiveness values of a palatable support composition disclosed herein can be measured, for example, by using a Texture Analyzer device fitted with a load cell, or probe, that moves up and down to compress and release the tested sample, and records the force response value of the tested sample to the deformation that is undergoing. Typically, data on force, distance and time are collected and are generally presented as a curve on a graph, which, when analyzed, provide the hardness value and the cohesiveness value that are indicative of the textural quality of the tested sample. Typically, hardness and cohesiveness values can be obtained through the same testing protocol, the information retrieved being available from the same set of data, which is known by the person skilled in the art. The hardness is generally the peak force at first compression, representing the force needed to break the sample, whereas the cohesiveness is the ratio of area of work under the second peak (second compression) divided by the area of work under the first peak (first compression), representing the ability of a sample to recover from first deformation. Cohesiveness is a dimensionless value (ratio). Illustratively, the Texture Analyzer can be a TA-HD Plus instrument commercialized by Texture Technologies (USA) company. The method used can be a Double Compression Texture Profile Analysis (TPA), using a 100-kg load cell, and a TA-40 compression probe (100 mm). The platform, or base, can be with reference TA-90. The measurement settings can be: Return Distance (mm): 0.5/Return Speed (mm/sec): 1/Contact Force (g): 50.

In some embodiments, a palatable support composition according to the present disclosure can have a hardness value from about 0.2 kg to about 1.0 kg or from about 0.2 kg to 0.6 kg. In certain embodiments, the palatable support composition can have a hardness value of about 0.2 kg, about 0.3 kg, about 0.4 kg, about 0.5 kg, about 0.6 kg, about 0.7 kg. about 0.8 kg, about 0.9 kg or about 1.0 kg. As it is known in the art, the hardness of a palatable support disclosed herein corresponds to the maximum force applied to break the said palatable support. The value of the hardness is currently used as a reference for characterizing the texture of a product.

In some embodiments, a palatable support composition according to the present disclosure can have a cohesiveness value from about 0.1 to about 0.5 or from about 0.2 to about 0.5 in a compression TPA test. In certain embodiments, the palatable support composition can have a cohesiveness value of about 0.1, about 0.2, about 0.3, about 0.4 or about 0.5 in a compression TPA test.

Without being bound by any theory, it is believed that the specific texture of the palatable support composition disclosed herein can be significantly imparted by the amount of glycerin, to which also contributes the combination and amounts of glycerin and gelling agent(s) comprised therein.

### Size, Dimensions, and Weight

The size and dimensions of the palatable support composition of the present disclosure can be adapted, for example, so as to comply with a “bite size”, i.e., a size and dimensions that can be adapted to be provided as such to an animal, such as a feline, without requiring to be divided or split before being provided to the animal.

In some embodiments, the palatable support composition can have a trapezoidal cylindrical shape. The trapezoidal cylindrical shape can include a bottom end and an upper end. The bottom end can be larger than the upper end. The bottom end can include a vertical axis and a horizontal axis. The vertical axis can pass through the bottom end and the upper end. Each of the bottom end and the upper end can define a circular surface plan, or alternatively an almost circular surface plan, that can be perpendicular to the vertical axis of the palatable support. The area of the circular surface plan values thereof can be determined by the diameters of the palatable support at the bottom end and at the upper end, respectively. The surface of each of (i) the bottom circular surface plan located at the bottom end; and (ii) the upper circular surface plan located at the upper end, can be flat, i.e., have a surface plan which is perpendicular to the vertical axis of the palatable support. The surface located at the upper end can show a cavity which allows the introduction of one or more medicinal products therein.

The size and dimensions of the palatable support composition can comply with a “bite size” (i.e., a size that is adapted to be given as such to an animal, in particular to a cat, without having to divide or split the palatable support prior to provide it to the animal). In some embodiments, the trapezoidal cylindrical support can have a height from about 5 mm to about 10 mm. In certain embodiments, a diameter of the trapezoidal cylindrical support can be from about 6 mm to about 12 mm at the bottom end. In certain embodiments, a diameter at the upper end of the trapezoidal cylindrical support can be from about 5 mm to about 10 mm.

In some embodiments, the palatable support composition can have a weight of from about 0.5 grams to about 2.0 grams, from about 1.0 grams to about 2.0 grams, from about 0.5 grams to about 1.0 grams, or from about 1.5 grams to about 2.0 grams. In certain embodiments, the palatable support composition can have a weight of about 0.5 grams, about 1.0 grams, about 1.5 grams, or about 2.0 grams.

### Palatability (Ingredients, Texture, and Size)

In certain embodiments, the high palatability properties of the palatable support compositions according to the present disclosure can be imparted, for example, (i) by the combination of ingredients included therein; (ii) by its texture properties; and (iii) by its size. The combination of ingredients can contribute to its good taste and flavor and to its ability to mask the possibly repulsing taste of the one or more medicinal products that are to be wrapped therein. Without being bound by any theory, it is believed that the specific combination of ingredients, including the specific combination of glycerin and protein, included in the palatable support compositions of the present disclosure can significantly contribute to the high palatability properties of the palatable support compositions disclosed herein. Its texture properties can contribute to its ease of prehension by the non-human mammal, for example, by the feline animal, and in particular by the cat. Its textural properties can also contribute to the adhesion of the said palatable support to the medicine put inside and allow the medicine to be ingested by the animal at the same time as the support, without the animal being able to separate the two. The optimal size of the said palatable support can also contributes to its easy grasping by the said non-human mammal.

### Moisture Content

In certain embodiments, the moisture content of a palatable support composition according to the present disclosure can be adapted, for example, so as to obtain a palatable support composition having the suitable texture properties allowing good palatability, such as a suitable chewiness rendering the palatable support attractive for the recipient feline. The moisture content of a palatable support as disclosed herein can also be adapted, for example, to lower the potential deleterious interactions with one or more moisture-sensitive ingredients contained therein. Thus, the moisture content of the palatable support composition can provide a balance between optimal texture properties of the palatable support and optimal preservation properties thereof all along shelf-life, with the view of limiting the possibility of mold development.

In certain embodiments, the palatable support composition can have a moisture content ranging from about 20% to about 35%, from about 25% to about 30%, from about 30% to about 35%, or from about 20% to about 25% as fed. In certain embodiments, the palatable support composition can have a moisture content of about 20%, about 21%, about 22%, about 23%, about 24%, about 25%, about 26%, about 27%, about 28%, about 29%, about 30%, about 31%, about 32%, about 33%, about 34% and about 35% as fed.

A moisture content of less than 20% can lead to a support material that is insufficiently adhering to the medicinal product(s) which it would have to hide. Such a support material would not be optimally adapted to molding and to wrapping a medicinal product, since it can break, or brittle while being used to wrap the medicinal product. In contrast, a moisture content of more than 35% can lead to a support material having an unacceptable softness and stickiness that would not be appreciated by the feline. Further, a high moisture content can be deleterious for one or more of the ingredients contained therein, particularly for moisture-sensitive ingredients such as vitamins. Excessive moisture can also lead to shelf-life issue such as molding.

The moisture content of any composition or product disclosed herein can be determined according to usual methods. The moisture content can conventionally be determined by (i) weighing the said composition or product; (ii) evaporating the free water contained therein, such as in an oven at a temperature ranging from about 100° C. to about 120° C. when performed at the atmospheric pressure, e.g., during a period of time ranging from about 1 hour to about 3 hours; and (iii) weighing the resulting dried composition or product, the weight difference consisting of the weight of water initially contained in the composition or product.

### 4.6. Applications and Kits

In certain embodiments, a palatable support composition according to the present disclosure is not intended to be used as a nutritionally complete food composition. Thus, in certain embodiments, the palatable support composition cannot be used as the sole composition for feeding a feline, and in particular a cat. However, according to certain embodiments, the palatable support composition as disclosed herein can be used as a food supplement beyond its use for facilitating the oral administration of one or more medicinal products to a feline in need thereof.

In certain embodiments, the palatable support composition disclosed herein can be a functional complement or a functional supplement. A functional complement or supplement is described as so because of its functional properties that differentiates it from a treat.

In certain aspects, the palatable support composition according to the present disclosure can be packaged in a pack containing a plurality of palatable support items, each palatable support item being possibly individually packaged itself. Thus, the present disclosure further provides a package including a plurality of palatable support items, each palatable support item being optionally individually packaged itself.

The presently disclosed palatable support compositions can be provided in one or more kits for consumer use. The kits can include, for example and not by way of limitation, one or more palatable support compositions of the present disclosure. The kits can also include one or more medicinal products. In one embodiment, the kit provides the palatable support composition separate from the one or more medicinal products. Optionally, such kids can further include any other systems components described in relation to the present disclosure and any other materials or items relevant to the present disclosure.

In certain embodiments, for example in embodiments wherein a prescribed medical product has a taste or a flavor that is repulsive for a feline, the medicinal product can be presented as included in a kit of parts including first and second parts. The first part can include the medicinal product. The second part can include a palatable support as disclosed herein. In certain aspects, the kit of parts can include (i) the first part which can include a container including a plurality of medicinal product dosage units (e.g., pills, tablets or capsules). Alternatively, the first part can include a container including an amount of a paste or of a gel corresponding to a plurality of prescribed dosage units. Further, the kit of parts can include (ii) the second part which can include a plurality of palatable support items according to the present disclosure. In certain aspects, the number of palatable support items can be at least in a sufficient number for administering all the medicinal product dosage units comprised in the said container. The number of palatable support items can be in excess in respect to the number of medicinal product dosage units contained in the container forming the first part of the kit. As such, the present disclosure further provides a kit of parts including (i) a palatable support as disclosed herein; and (ii) one or more medicinal products or product dosage units.

### 5. EXAMPLES

The presently disclosed subject matter will be better understood by reference to the following Examples, which are provided as exemplary of the disclosure, and not by way of limitation. The following Examples are merely illustrative of the presently disclosed subject matter and they should not be considered as limiting the scope of the subject matter in any way.

### Example 1: Manufacture of Palatable Support Compositions

The present Example provides processes for making palatable support compositions in accordance with the present disclosure.

### A. Materials and Manufacturing Process

In a first step, all dry ingredients were mixed and homogenized together. Then, liquids were added (fats, water and glycerin) to the dry mix in order to form a dough. The dough was cooked and pasteurized (min. 90° C.) in order to melt the ingredients together into the desired texture and to ensure its stability over time. The hot dough was then cooled down and pumped into individual molds to provide their final dimension. Each individual piece was at its final stage and was packed before being stored away at ambient temperature.

The ingredients and their proportions used for manufacturing a palatable support composition in accordance with the present disclosure is provided in Table 1 below.

TABLE 1
Palatable Support Composition Ingredients

| Ingredients | Percent (w/w) |
|---|---|
| Cereals (flour) | 14.0% |
| Animal proteins | 21.4% |
| Vegetable protein | 6.0% |
| Corn syrup solids | 2.5% |
| Preservatives and antioxidants | 0.6% |
| Gelling agent | 2.0% |
| Acidifier | 0.5% |
| Prebiotics and vitamins | 0.8% |
| Palatability enhancer | 2.0% |
| Yeast Extract | 2.0% |
| Glycerin | 23% |
| Water and steam condensates | 22% |
| Vegetable fats | 3.2% |
| Total | 100% |

Palatable support compositions of the present disclosure were prepared as follows.

a) Preparation of the dry mix blend. All dry ingredients, cereals, animal and vegetable proteins, corn syrup, preservatives and antioxidants, gelling agent, acidifier, prebiotics and vitamins, yeast extract and palatability enhancer, were weighed and homogeneously mixed together until a powdered pre-blend was obtained, containing all the nutritional elements.

b) Preparation of a glycerin-containing blend. The aqueous elements, glycerin and water, in a ratio of about 60:40 glycerin:water, were mixed together in a tank.

c) Preparation of a fat blend. Vegetable fats were mixed together in a separate heated tank (non-aqueous components).

d) Mixing and cooking to form a dough. The dry mix blend, the glycerin-containing blend, and the fat blend were all used to feed a continuous dough cooking apparatus (Reading system) at respective amounts of 51.8% w/w dry mix, 37% w/w glycerin pre-blend, and 3.2% w/w fat blend. While mixing, culinary steam was injected in the Reading cooker, in an amount of 8% w/w, until pasteurizing temperature of 90° C. was reached. The product that was obtained corresponded to a smooth, uniform dough where all ingredients were blended and melted together. As the Reading system operates on continuous mode, the undercooked dough was not allowed to move to further stages of the process, and goes to a trash bin. Standard operating conditions allow the production of a dough at a rate of about 250 kg/h.

e) Cooling of the dough. Prior to forming, the dough was cooled. The cooling step is critical: hard enough to have the right texture for forming and not collapsing, but still soft enough to be pumped into the small individual molds. Cooling was done on a continuous stainless-steel belt, chilled underneath by a system with cold water recirculation.

f) Molding of the cooled dough. When cool enough, the dough was be pumped into small individual molds corresponding to the final shape and dimensions of the support. Individual pieces were expulsed from their molds and conveyed on belt until packing equipment.

### B. Palatable Support Compositions

The composition of the final palatable support composition prepared by the method above is provided in Table 2 below.

TABLE 2
Palatable Support Compositions

| Composition | Percent | Percent on dry |
|---|---|---|
| (average values) | (w/w) | matter basis (w/w) |
| Moisture | 27.0% | N/A |
| Protein | 21.0% | 29.0% |
| Fat Content | 12.5% | 17.0% |
| Crude Ash | 1.8% | 2.5% |
| Crude Fiber | 1.1% | 1.5% |
| Nitrogen-Free Extract (NFE) | 36.6% | 50.0% |
| Total | 100.0% | 100.0% |

### Example 2: Palatability Testing of Palatable Support Compositions

The present Example provides for palatability testing between two (2) formulations of palatable support compositions (Product A and Product B) according to the present disclosure and prepared in accordance with Example 1. Preference between the two (2) palatable support compositions was assessed. Compositions of the two (2) palatable support compositions tested (Product A and Product B) are provided in Table 3 below. Product A included chicken as a protein source and Product B included salmon as a protein source.

TABLE 3
Compositions of Products A and B

| Composition—average values, in | | |
|---|---|---|
| percent on dry matter basis (w/w) | Product A | Product B |
| Moisture | N/A | N/A |
| Protein | 29.0% | 32.0% |
| Fat Content | 17.0% | 16.5% |
| Crude Ash | 2.5% | 3.5% |
| Crude Fiber | 1.5% | 0.4% |
| Nitrogen-Free Extract (NFE) | 50.0% | 47.6% |
| Total | 100.0% | 100.0% |

Product A (chicken): Hardness value: 0.33 kg/Cohesiveness value: 0.18 (dimensionless)
Product B (salmon): Hardness value: 0.24 kg/Cohesiveness value: 0.18 (dimensionless)

### A. Materials and Methods

The assessment was conducted in a panel consisting of 31 cats, the animals being presented two bowls at the same time.

### Pre-Exposure

Before the actual assay and during a 3-day period, the test samples as well as a positive control (very palatable treat from the US market) were fed to the cats in a monadic way, with cross-over design, twice per day, in order to get them used to the texture. The first exposure of the day was after their first wet meal and as close as possible to their second wet meal and the second exposure was given to the cats after their second wet meal and as close as possible to their dry meal. Exposure time was 5 minutes. The pre-exposure routine is provided in Table 4 below.

TABLE 4
Example Pre-Exposure

| | Meal 1 | Meal 2 |
|---|---|---|
| Group A— | Bowl 1: 1 control piece | Bowl 1: 1 piece of either |
| 16 cats | Bowl 2: 1 piece of either | product A OR product B |
| | product A OR product B | Bowl 2: 1 control piece |
| Group B— | Bowl 1: 1 piece of either | Bowl 1: 1 control piece |
| 15 cats | product A OR product B | Bowl 2: 1 piece of either |
| | Bowl 2: 1 control piece | product A OR product B |

### Assay

The assessment was conducted in a panel consisting of 31 cats, the animals being presented two bowls at the same time. A crossover design was used to ensure bowl position did not bias the outcome of the trials. All tests were performed in the cage banks. The products were given to the cats after their second wet meal and as close as possible to their evening dry meal (therefore 1 distribution per day). Each cat received 5 product units per bowl. Exposure time was 10 minutes. The distribution was repeated on the second day with a crossover on bowl distribution. The crossover model is provided in Table 5 below.

TABLE 5
Crossover Model (2 days)

| | Day 1 | | Day 2 | |
|---|---|---|---|---|
| Animals | Left | Right | Left | Right |
| Pets | Product A | Product B | Product B | Product A |
| 1-31 | | | | |

### B. Results

Results are expressed as first bowl finished, with a 95% confidence interval. The results are provided in Table 6 below. Product A was preferred to Product B. Product A was selected for further Examples provided below.

TABLE 6
Results

| Expected Number | 31 cats | | |
|---|---|---|---|
| Validated Number | 31 cats | | |
| Preference | A: 47.5% | B: 13.2% | NC: 39.34% |

| | |
|---|---|
| Preference | 0.0006 (VHS) |
| Probability | |
| Conclusion | Product A is very highly significantly |
| | more preferred than Product B. |

### Example 3: Monadic Test to Assess Digestive Tolerance

The present Example provides for digestive tolerance testing of palatable support compositions of the present disclosure.

### A. Materials and Methods

A monadic test was performed to measure the digestive tolerance of Product A of Example 2 and manufactured according to Example 1 to the same panel (n=31) of cats. During the 5-day test, the cats only received monadic product testing and no other testing. Cats received their regular meals of wet and dry foods. The composition of Product A tested in Example 3 is provided in Table 7 below.

TABLE 7
Product Composition (Product A)—Example 3

| Composition—average values, | |
|---|---|
| in percent on dry matter (w/w) | Product A |
| Moisture | N/A |
| Protein | 29.0% |
| Fat Content | 17.0% |
| Crude Ash | 2.5% |
| Crude Fiber | 1.5% |
| Nitrogen-Free Extract (NFE) | 50.0% |
| Total | 100.0% |

Product A: Hardness: 0.33 kg/Cohesiveness: 0.18 (dimensionless)

The first exposure was given to the cats after the first wet meal and as close as possible to their second wet meal and the second exposure was given to the cats after the second wet meal and as close as possible to their dry meal and so on. Exposure time was 5 minutes. The product was offered in a bowl, and if not taken it was offered by hand, or left on the floor of the cage.

Litterbox stool scores were collected at the end of each day. Stool quality were assessed on a 5-point scale, from 1 to 5, 1 being very dry stool to 5 being very wet and soft stools. The optimal score was at 2.5.

### B. Results

The results are provided in Table 8 below. Room litterbox fecal scores were considered close enough to optimal scores, and so the digestive tolerance test was validated.

TABLE 8
Results
Analysis Variable: score

| Mean | Std Dev | Minimum | Maximum |
|---|---|---|---|
| 2.46 | 1.55 | 1.75 | 3.00 |

### Example 4: Monadic Test to Assess Product Acceptance

The present Example provides for product acceptance testing of palatable support compositions of the present disclosure.

### A. Materials and Methods

A monadic test was performed to measure the overall product acceptance of Product A of Example 2 and manufactured according to Example 1 to the same panel (n=31) of cats. The acceptance was assessed either with a placebo drug inside or without. During the 5-day test, the cats only received monadic product testing and no other testing. Cats received their regular meals of wet and dry foods. The composition of Product A tested in Example 4 is provided in Table 9 below.

TABLE 9
Product Composition (Product A)—Example 4

| Composition—average values, | |
|---|---|
| in percent on dry matter (w/w) | Product A |
| Moisture | N/A |
| Protein | 29.0% |
| Fat Content | 17.0% |
| Crude Ash | 2.5% |
| Crude Fiber | 1.5% |
| Nitrogen-Free Extract (NFE) | 50.0% |
| Total | 100.0% |

Product A: Hardness value: 0.33 kg/Cohesiveness value: 0.18 (dimensionless)

The part corresponding to “without placebo” is detailed in Example 3: the fecal scores were assessed, but the acceptance of the product was also rated. The same panel (n=31) of cats was used to perform the second part “with placebo”. The objective of using a placebo inside was to prove the ability to hide a drug/medicine under real usage conditions. The drug placebo used were Torpac® #5 0.13 ml gelatin capsules. The product “with placebo” was prepared as described herein: the moldable palatable support was handled with one hand, i.e., the hand that touched the medicine was not used to wrap and close the mass around it.

The first exposure was given to the cats after the first wet meal and as close as possible to their second wet meal and the second exposure was given to the cats after the second wet meal and as close as possible to their dry meal and so on. Exposure time was 5 minutes. The product was offered in a bowl, and if not taken it was offered by hand, or left on the floor of the cage. In the end, cats were offered the products twice a day for 5 days, i.e., 10 exposures per cat and 310 exposures in total, the overall repeated a second time with the placebo.

### B. Results

### Part I: No Placebo

During the test presented in Example 3, where fecal scores were recorded, overall acceptance of the product given was also recorded. The results are provided below in Table 10. The results in Table 10 show the acceptance rate over the 310 exposures that were afterwards analyzed with Clopper-Pearson (Exact) test at 95% confidence interval level.

TABLE 10
Results (No Placebo)

| | | | Cumulative | Cumulative |
|---|---|---|---|---|
| Accepted | Frequency | Percent | Frequency | Percent |
| No | 32 | 10.32 | 32 | 10.32 |
| Yes | 278 | 89.68 | 310 | 100.00 |

In conclusion, when given (no placebo) twice daily for 5 days, the product detailed in the Material and Methods section showed a mean acceptance of 89.68% with a very highly significant confidence (p=0.0001).

### Part II. With Placebo

Results below show the acceptance rate over the 310 exposures of the product with placebo wrapped inside, that were afterwards analyzed with Clopper-Pearson (Exact) test at 95% confidence interval level, provided in Table 11 below.

TABLE 11
Results (With Placebo)

| | | | Cumulative | Cumulative |
|---|---|---|---|---|
| Accepted | Frequency | Percent | Frequency | Percent |
| No | 23 | 7.42 | 23 | 7.42 |
| Yes | 287 | 92.58 | 310 | 100.00 |

In conclusion, when given with a gelatin capsule placebo wrapped inside twice daily for 5 days, the product detailed in the Material and Methods section showed a mean acceptance of 92.58% with a very highly significant confidence (p=0.0001). When combining all exposures, i.e., either with or without placebo inside, the results of overall mean acceptance using Clopper-Pearson (Exact) test at 95% confidence interval level, shows the results in Table 12 below.

TABLE 12
Results (With or Without Placebo)

| | | | Cumulative | Cumulative |
|---|---|---|---|---|
| Accepted | Frequency | Percent | Frequency | Percent |
| No | 55 | 8.87 | 55 | 8.87 |
| Yes | 565 | 91.13 | 620 | 100.00 |

In conclusion, when given twice daily for 5 days then twice daily for 5 days with a gelatin capsule placebo wrapped inside (total 620 exposures), the product detailed in the Material and Methods section showed a mean acceptance of 91.13% with a very highly significant confidence (p=0.0001).

Although the presently disclosed subject matter and its advantages have been described in detail, it should be understood that various changes, substitutions and alterations can be made herein without departing from the spirit and scope of the application as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

For any patents, patent applications, publications, product descriptions, and protocols are cited throughout this application, the disclosures of all of which are incorporated herein by reference in their entireties for all purposes.

1. A palatable support composition for administering one or more medicinal products to a feline, comprising:
(i) from about 20% to about 40% by weight of a humectant plasticizer;
(ii) from about 0.5% to about 8% by weight of one or more gelling agents; and (iii) from about 20% to about 50% by weight of protein,
the weight percentage being expressed on a dry matter basis, and
the palatable support composition having a moisture content from about 20% to about 35% as fed.

2. The composition according to claim 1, wherein the humectant plasticizer is glycerin.

3. The composition according to claim 1, wherein the composition has a hardness value from about 0.2 kg to about 1.0 kg and a cohesiveness value from about 0.1 to about 0.5 in a compression TPA test.

4. The composition according to claim 1, wherein the composition has a trapezoidal cylindrical shape with a bottom end and an upper end, a height from about 5 mm to about 10 mm, a diameter from about 6 mm to about 12 mm at the bottom end, and a diameter from about 5 mm to about 10 mm at the upper end.

5. The composition according to claim 1, wherein the composition has a weight from about 0.5 grams to about 2.0 grams.

6. The composition according to claim 1, wherein the one or more gelling agents comprise gum Arabic, xanthan gum, guar gum, locust bean gum, carob gum, carrageenan, or a combination thereof.

7. The composition according to claim 1, wherein the composition further comprises fat in an amount from about 7.5% to about 20% by weight, on a dry matter basis.

8. The composition according to claim 1, wherein the protein originates from one or more protein sources comprising chicken, poultry, fish, or a combination thereof.

9. The composition according to claim 1, further comprising one or more of the following:
i) from about 1% to about 8% by weight yeast extract, on a dry matter basis;
ii) from about 1% to about 8% by weight one or more palatability enhancers, on a dry matter basis; and
iii) from about 1% to about 8% by weight corn syrup solids, on a dry matter basis.

10. The composition according to claim 1, wherein the composition is a functional supplement.

11. The composition according to claim 1, wherein the feline is a cat.

12. A kit, comprising: (i) a palatable support composition according to claim 1; and (ii) one or more medicinal product dosage units.

13. Use of a palatable support composition according to claim 1 for facilitating a feline medication.

14. Use of a palatable support composition according to claim 1 for administering one or more medicinal products to a feline.

15. A method of use of a palatable support composition according to claim 1 for administering one or more medicinal products to a feline, comprising:
a) selecting one or more medicinal products to be administered;
b) incorporating the one or more medicinal products in the palatable support composition to provide an incorporated medicinal product; and
c) providing the incorporated medicinal product to the feline.

16. A method of use of a palatable support composition for administering one or more medicinal products to a feline, comprising:
a) selecting one or more medicinal products to be administered;
b) incorporating the one or more medicinal products in the palatable support to provide an incorporated medicinal product; and
c) providing the incorporated medicinal product to the feline,
wherein the palatable support composition comprises:
(i) from about 20% to about 40% by weight humectant plasticizer;
(ii) from about 0.5% to about 8% by weight of one or more gelling agents; and
(iii) from about 20% to about 50% by weight of protein,
the weight percentage being expressed on a dry matter basis, and
the palatable support composition having a moisture content ranging from about 20% to about 35% as fed.

17. A method comprising providing a palatable support composition for administering one or more medicinal products to a feline, the palatable support composition comprising (i) from about 20% to about 40% by weight humectant plasticizer; (ii) from about 0.5% to about 8% by weight of one or more gelling agents; and (iii) from about 20% to about 50% by weight of protein, the weight percentage being expressed on a dry matter basis, and the palatable support having a moisture content ranging from about 20% to about 35% as fed.

18. A method for administering one or more medicinal products to a feline, comprising providing a palatable support composition comprising (i) from about 20% to about 40% by weight humectant plasticizer; (ii) from about 0.5% to about 8% by weight of one or more gelling agents; and (iii) from about 20% to about 50% by weight of protein, the weight percentage being expressed on a dry matter basis, and the palatable support having a moisture content ranging from about 20% to about 35% as fed.

19. A method according to claim 15, wherein the feline is a cat.

20. A method for preparing a palatable support composition according to claim 1, comprising:
a) providing a blend of a plurality of dry ingredients including proteins;
b) providing an aqueous solution including a humectant plasticizer;
c) providing a fat mixture, comprising a plurality of fat-based ingredients from animal and/or vegetable origin;
d) mixing the blend of the plurality of dry ingredients, the aqueous solution, and the fat mixture, and heating the resulting mixture to a temperature of at least about 80° C. to provide a cooked dough;
e) cooling the cooked dough; and
f) molding the cooled dough to provide the palatable support composition.
